(19) United States
(12) Patent Application Publication
(10) Pub. No.: US 2024/0197189 A1
(43) Pub. Date: Jun. 20, 2024

(54) BIOLOGICAL INFORMATION MEASUREMENT DEVICE

(71) Applicant: OMRON HEALTHCARE CO., LTD, Kyoto (JP)

(72) Inventors: Kentaro MORI, Kyoto (JP); Takeshi KUBO, Kyoto (JP); Takashi ONO, Kyoto (JP); Yoshihide TOKKO, Kyoto (JP); Masaki HARADA, Kyoto (JP); Kosuke ABE, Kyoto (JP); Shohei IWATA, Kyoto (JP); Yoshihiko SANO, Kyoto (JP)

(73) Assignee: OMRON HEALTHCARE CO., LTD, Kyoto (JP)

(21) Appl. No.: 18/592,574

(22) Filed: Mar. 1, 2024

Related U.S. Application Data
(63) Continuation of application No. PCT/JP2023/019189, filed on May 23, 2023.

(30) Foreign Application Priority Data
Sep. 5, 2022 (JP) 2022-140478

(51) Int. Cl. A61B 5/021; A61B 5/0205; A61B 5/024; A61B 5/0245

(52) U.S. Cl. CPC A61B 5/02141; A61B 5/0205; A61B 5/02427; A61B 5/02438; A61B 5/0245; A61B 2560/0468; A61B 2562/168

(57) ABSTRACT

A biological information measurement device measuring blood pressure of a subject and biological information which includes: a curved support member; a belt portion; a fluid bag; a pump; a valve; a blood pressure measurement unit; a detection unit which comes into contact with the measurement site of the subject; and a biological information measurement unit measuring the biological information with the detection unit, in which the fluid bag includes a first bag portion and a second bag portion, and the detection unit which is arranged at a position which allows a change in a contact state between the detection unit and the measurement target site due to movement of the detection unit support portion accompanying a volume change of the fluid bag and the second bag portion to be limited to a predetermined range.

### CROSS-REFERENCE TO RELATED APPLICATION

This application is the U.S. national stage application filed pursuant to 35 U.S.C. 365(c) and 120 as a continuation of International Patent Application No. PCT/JP2023/019189, filed on May. 23, 2023, which application claims priority to Japanese Patent Application No. 2022-140478, filed on Sep. 5, 2022, which applications are incorporated herein by reference in their entireties.

### TECHNICAL FIELD

The present invention relates to a biological information measurement device.

### BACKGROUND ART

In recent years, it has been common for an individual to measure his/her body/health information (hereinafter, also referred to as biological information) such as an electrocardiographic waveform together with a blood pressure value by using a measurement instrument on a daily basis and utilize the measurement result for health management. For this reason, there is an increasing demand for instruments focusing on portability, many portable measurement devices have been proposed, and a portable instrument capable of measuring both the blood pressure value and the electrocardiographic waveform has also been proposed (see Patent Literatures 1 and 2).

In Patent Literature 1, an electrode is arranged on a back surface (a surface in contact with a user's body) of a main body of a wristwatch-type electrocardiograph.

In Patent Literature 2, an electrode is arranged on a front surface (a surface in contact with a user's body) of a belt-shaped cuff wound around an arm of the user.

### CITATION LIST

### Patent Literatures

Patent Literature 1: JP 2017-6230 A

Patent Literature 2: JP 2014-36843 A

### SUMMARY OF INVENTION

### Technical Problem

However, in a configuration in which the electrode is arranged on the back surface of the main body as in the technique described in Patent Literature 1, the cuff cannot be arranged on the back surface of the main body, and thus, a compressing force on the user for blood pressure measurement is insufficient. As in the technique described in Patent Literature 2, in a configuration in which the electrode is arranged on the surface of the cuff, it is difficult to wire the electrodes, and it is necessary to design the wire in consideration of the movement of the electrode accompanying the inflation of the cuff, resulting in a complicated configuration in design. In addition, in a case where a rigid electrode is used, the compression of an air bladder provided in the cuff is hindered.

In view of the above-described conventional techniques, an object of the present invention is to provide a biological information measurement device capable of stably measuring blood pressure and biological information with a simple configuration.

### Solution to Problem

In order to solve the above problems, the present invention provides a biological information measurement device which measures blood pressure of a subject and biological information different from the blood pressure, the biological information measurement device including:
a belt portion which is wound around an outer circumference of a measurement target site of the subject and fixes the biological information measurement device to the measurement site;
a fluid bag which is arranged on an inner circumferential side of the support member;
a pump which supplies a fluid into the fluid bag;
a valve which is provided in a flow path of the fluid communicating with the fluid bag;
a blood pressure measurement unit which compresses the measurement target site by supplying the fluid from the pump to inflate the fluid bag or releases compression on the measurement target site by controlling the valve to discharge the fluid in the fluid bag to contract the fluid bag, and measures a blood pressure of the measurement target site;
a detection unit which comes into contact with the measurement site of the subject and is configured to measure the biological information; and
a biological information measurement unit which measures the biological information by using the detection unit,
in which the fluid bag includes a first bag portion and a second bag portion which are arranged apart from each other in a circumferential direction of the support member and communicate with each other, and
the detection unit
   is supported by a detection unit support portion which is the support member exposed to an inner circumferential side between the first bag portion and the second bag portion, and
   is arranged at a position which allows a change in a contact state between the detection unit and the measurement target site due to movement of the detection unit support portion accompanying a volume change of the fluid bag and the second bag portion to be limited to a predetermined range, the position being a position separated by a first predetermined distance from a first end portion of the first bag portion on the detection unit side and separated by a second predetermined distance from a first end portion of the second bag portion on the detection unit side.

Accordingly, in the detection unit to be used to measure biological information different from the blood pressure of the subject, when the fluid bag arranged on the inner circumferential side of the support member is inflated in a state where the biological information measurement device is fixed to the measurement target site for the blood pressure measurement of the subject by the belt portion wound around the outer circumference of the measurement target site of the subject, the detection unit is supported by the detection unit support portion exposed to the inner circumferential side between the first bag portion and the second bag portion arranged apart from each other in the circumferential direction of the support member, and the detection unit is arranged at a position which allows a change in the contact state between the detection unit and the measurement target site due to the movement of the detection unit support portion accompanying the volume change of the first bag portion and the second bag portion to be limited to a predetermined range, the position being a position separated by the first predetermined distance from the first end portion of the first bag unit on the detection unit side and separated by the second predetermined distance from the first end portion of the second bag unit on the detection unit side. Therefore, even when the support member moves to the outer diameter side along with the inflation of the first bag portion and the second bag portion, the change in the contact state between the detection unit and the measurement target site is limited to the predetermined range, so that the biological information can be stably measured, and the blood pressure measurement is not hindered, and thus, stable measurement of the blood pressure and the biological information can be realized with a simple configuration that is easy to manufacture.

Furthermore, in the present invention, the detection unit may be a photoplethysmography sensor, and the biological information may be a pulse wave or information based on the pulse wave.

As described above, by using the photoplethysmography sensor as the detection unit, stable measurement of the blood pressure and the pulse wave or information based on the pulse wave can be realized with a simple configuration that is easy to manufacture.

In addition, in the present invention, the detection unit may be an arterial oxygen saturation sensor, and the biological information may be arterial oxygen saturation or information based on the arterial oxygen saturation.

As described above, by using the arterial oxygen saturation sensor as the detection unit, stable measurement of the blood pressure and the arterial oxygen saturation or the information based on the arterial oxygen saturation can be realized a simple configuration that is easy to manufacture.

In addition, in the present invention, the biological information may be an electrocardiographic waveform, and
the biological information measurement device may further include:
   a first electrode which comes into contact with a first site of the subject;
   a second electrode which is the detection unit and comes into contact with a second site which is the measurement target site different from the first site;
   an electrode support portion which is the detection unit support portion; and
   an electrocardiogram measurement unit which is the biological information measurement unit and measures the electrocardiographic waveform of the subject through the first electrode and the second electrode.

Such a biological information measurement device includes: a first electrode which comes into contact with a first site of a subject; a second electrode which comes into contact with a second site which is a measurement target site different from the first site and is supported by an electrode support portion; and an electrocardiogram measurement unit which measures an electrocardiographic waveform of the subject through the first electrode and the second electrode. In this biological information measurement device, the second electrode is supported by the electrode support portion of the detection member exposed to the inner circumferential side between the first bag portion and the second bag portion arranged apart from each other in the circumferential direction of the support member, and is arranged at a position which allows the change in the contact state between the second electrode and the measurement target site due to the movement of the support member accompanying the volume change of the first bag portion and the second bag portion to be limited to a predetermined range. Therefore, even when the support member moves to the outer diameter side along with the inflation of the first bag portion and the second bag portion, the change in the contact state between the second electrode and the second site is limited to the predetermined range, so that the electrocardiographic waveform can be stably measured, and the blood pressure measurement is not hindered, and thus, stable measurement of the blood pressure and the electrocardiographic waveform can be realized with a simple configuration that is easy to manufacture.

In addition, in the present invention, the biological information measurement device may include a third electrode which comes into contact with a third site of the subject different from the first site and the second site and sets a reference potential, in which
the third electrode may
   be supported by an extension portion of the support member extending, to a side opposite to the first end portion, from a second end portion of the second bag portion on a side opposite to the first end portion, and
   be arranged at a position which allows a change in a contact state between the third electrode and the third site due to movement of the extension portion accompanying a volume change of the second bag portion to be limited to a predetermined range, the position being a position separated by a third predetermined distance from the second end portion of the second bag portion.

Accordingly, the biological information measurement device further includes the third electrode which sets the reference potential when the electrocardiographic waveform of the subject is measured through the first electrode and the second electrode, so that the electrocardiographic waveform can more accurately be measured. In addition, the third electrode is supported by the extension portion of the support member extending, to a side opposite to the first end portion, from the second end portion of the second bag portion on a side opposite to the first end portion, and is arranged at a position which allows the change in the contact state between the third electrode and the third site due to the movement of the extension portion accompanying the volume change of the second bag portion to be limited to the predetermined range, the position being a position separated by the third predetermined distance from the second end portion of the second bag portion. Therefore, even when the support member moves to the outer diameter side along with the inflation of the second bag portion, the change in the contact state between the third electrode and the third site is limited to the predetermined range, so that the electrocardiographic waveform can be stably measured, and the blood pressure measurement is not hindered, and thus, stable measurement of the blood pressure and the electrocardiographic waveform can be realized with a simple configuration that is easy to manufacture.

In addition, in the present invention, the biological information measurement device may include a third electrode which comes into contact with a third site of the subject different from the first site and the second site and sets a reference potential,
in which the third electrode may be supported by the electrode support portion, and be arranged at a same position as the second electrode in the circumferential direction with respect to the first bag portion and the second bag portion and at a position aligned with the second electrode in a direction orthogonal to the circumferential direction.

Accordingly, the biological information measurement device further includes the third electrode which sets the reference potential when the electrocardiographic waveform of the subject is measured through the first electrode and the second electrode, so that the electrocardiographic waveform can more accurately be measured. In addition, the third electrode is supported by the electrode support portion and is arranged at the same position as the second electrode in the circumferential direction with respect to the first bag portion and the second bag portion and at the position aligned with the second electrode in the direction orthogonal to the circumferential direction, so that even when the support member moves to the outer diameter side along with the inflation of the first bag portion and the second bag portion, the change in the contact state between the third electrode and the third site is limited to the predetermined range, so that the electrocardiographic waveform can be stably measured, and the blood pressure measurement is not hindered, and thus, stable measurement of the blood pressure and the electrocardiographic waveform can be realized with a simple configuration that is easy to manufacture.

Further, in the present invention, the biological information measurement device may include: a fourth electrode which comes into contact with a fourth site of the subject different from the first site and the second site,
in which the electrocardiogram measurement unit may measure the electrocardiographic waveform of the subject through the first electrode, the second electrode, and the fourth electrode,
the fluid bag may further include a third bag portion which is arranged apart from the second bag portion on a side opposite to the first bag portion in the circumferential direction of the support member and communicates with the second bag portion, and
the fourth electrode may be supported by a second electrode support portion which is the support member exposed to an inner circumferential side between the second bag portion and the third bag portion, and be arranged at a position which allows a change in a contact state between the fourth electrode and the fourth site due to movement of the second electrode support portion accompanying a volume change of the second bag portion and the third bag portion to be limited to a predetermined range, the position being a position separated by a fourth predetermined distance from a second end portion of the second bag portion on the fourth electrode side and separated by a fifth predetermined distance from a first end portion of the third bag portion on the fourth electrode side.

In this way, the fourth electrode to be used for measurement of the electrocardiographic waveform, together with the first electrode and the second electrode, is supported by the second electrode support portion of the support member exposed to the inner circumferential side between the third bag portion and the second bag portion provided on the side opposite to the first bag portion in the circumferential direction with respect to the second bag portion, and is arranged at a position which allows the change in the contact state between the fourth electrode and the fourth site due to the movement of the second electrode support portion accompanying the volume change of the second bag portion and the third bag portion to be limited to the predetermined range, the position being a position separated by the fourth predetermined distance from the second end portion of the second bag portion on the fourth electrode side and separated by the fifth predetermined distance from the first end portion on the fourth electrode side of the third bag portion, and thus, even when the support member including the second electrode support portion moves to the outer diameter side along with the inflation of the second bag portion and the third bag portion, the change in the contact state between the fourth electrode and the fourth site is limited to the predetermined range, and the electrocardiographic waveform can be stably measured. In addition, according to the biological information measurement device, the blood pressure measurement is not hindered, and thus stable measurement of the blood pressure and the electrocardiographic waveform can be realized with a simple configuration that is easy to manufacture.

In the present invention, the fourth electrode may be electrically connected to the second electrode.

As described above, when the second electrode and the fourth electrode are electrically connected to each other, the contact area of the second electrode with the measurement site of the subject is substantially increased, so that more stable measurement of the electrocardiographic waveform can be performed.

In addition, in the present invention,
the fourth electrode may not be electrically connected to the second electrode, and
the electrocardiogram measurement unit may measure the electrocardiographic waveform on the basis of any one of a first potential difference detected by the first electrode and the second electrode or a second potential difference detected by the first electrode and the fourth electrode.

Accordingly, when there is a difference between the quality of the electrocardiographic waveform measured on the basis of the first potential difference and the quality of the electrocardiographic waveform measured based on the second potential difference, or when there is a difference between the contact resistances of a pair of the first electrode and the second electrode and a pair of the first electrode and the fourth electrode, the electrocardiographic waveform can be measured by selecting the potential difference of a relatively excellent pair, so that more accurate electrocardiographic waveform measurement can be performed.

In addition, in the present invention, the biological information measurement device may include:
a third electrode which comes into contact with a third site of the subject different from any one of the first site, the second site, and the fourth site and sets a reference potential; and
a fifth electrode which comes into contact with a fifth site of the subject different from any one of the first site, the second site, the third site, and the fourth site and is electrically connected to the third electrode,
in which the third electrode may be supported by the electrode support portion, and be arranged at a same position as the second electrode in the circumferential direction with respect to the first bag portion and the second bag portion and at a position aligned with the second electrode in a direction orthogonal to the circumferential direction, and
the fifth electrode may be supported by the second electrode support portion, and be arranged at a same position as the fourth electrode in the circumferential direction with respect to the second bag portion and the third bag portion and at a position aligned with the fourth electrode in a direction orthogonal to the circumferential direction.

Accordingly, the biological information measurement device further includes the third electrode and the fifth electrode which set the reference potential when the electrocardiographic waveform of the subject is measured through the first electrode, the second electrode, and the fourth electrode, the electrocardiographic waveform can be more accurately measured. In addition, the third electrode is supported by the electrode support portion and is arranged at the same position as the second electrode in the circumferential direction with respect to the first bag portion and the second bag portion and at the position aligned with the second electrode in the direction orthogonal to the circumferential direction, so that even when the support member including the detection support portion moves to the outer diameter side along with the inflation of the first bag portion and the second bag portion, the change in the contact state between the third electrode and the third site is limited to the predetermined range. In addition, the fifth electrode is supported by the second electrode support portion and is arranged at the same position as the fourth electrode in the circumferential direction with respect to the second bag portion and the third bag portion and at the position aligned with the fourth electrode in the direction orthogonal to the circumferential direction, so that even when the support member including the second electrode support portion moves to the outer diameter side along with the inflation of the second bag portion and the third bag portion, the change in the contact state between the fifth electrode and the fifth site is limited to the predetermined range. Therefore, the electrocardiographic waveform can be stably measured and the blood pressure measurement is not hindered, so that stable measurement of the blood pressure and the electrocardiographic waveform can be realized with a simple configuration that is easy to manufacture.

In addition, in the present invention,
the support member may be a curler curved along a circumferential direction of the measurement target site of the subject, and
the belt portion may be wound around an outer circumferential side of the curler.

In this way, the fluid bag or the like is arranged on the inner circumferential side of the curler curved in advance along the circumferential direction of the measurement target site of the subject, and the belt portion is wound from the outer circumferential side of the curler, so that the biological information measurement device can be easily and reliably worn at an appropriate position with respect to the measurement target site.

In the present invention, the curler may be joined to the belt portion.

By joining the belt portion to the curler in this manner, the biological information measurement device can be easily worn.

In addition, in the present invention, the support member may constitute a part of the belt portion.

As described above, when the support member constitutes a part of the belt portion, and the belt portion also serves as the support member, the configuration of the biological information measurement device can be simplified, and handling is also facilitated.

### Advantageous Effects of Invention

According to the present invention, it is possible to provide a biological information measurement device capable of stably measuring blood pressure and biological information with a simple configuration.

### BRIEF DESCRIPTION OF DRAWINGS

FIG. 1 is a view illustrating an appearance of a biological information measurement device according to a first embodiment.

FIG. 2 is a view illustrating an appearance when the biological information measurement device according to the first embodiment is worn.

FIG. 3 is a functional block diagram of the biological information measurement device according to the first embodiment.

FIGS. 4A and 4B are views illustrating a use state of the biological information measurement device according to the first embodiment.

FIGS. 5A and 5B are a side view and a rear view of the biological information measurement device according to the first embodiment.

FIGS. 6A and 6B are a side view and a rear view of the biological information measurement device according to the first embodiment.

FIGS. 7A and 7B are a side view and a rear view of a biological information measurement device according to a modification of the first embodiment.

FIGS. 8A and 8B are a side view and a rear view of a biological information measurement device according to a second embodiment.

FIG. 9 is a functional block diagram of the biological information measurement device according to the second embodiment.

FIG. 10 is a functional block diagram of a biological information measurement device according to a modification of the second embodiment.

### DESCRIPTION OF EMBODIMENTS

Hereinafter, specific embodiments of the present invention will be described on the basis of the drawings.

### First Embodiment

Hereinafter, an embodiment of an embodiment of the present invention will be described. However, the dimensions, materials, shapes, relative arrangements, and the like of the components described in this embodiment are not intended to limit the scope of the present invention only to them unless otherwise specified.

### Overall Configuration of Device

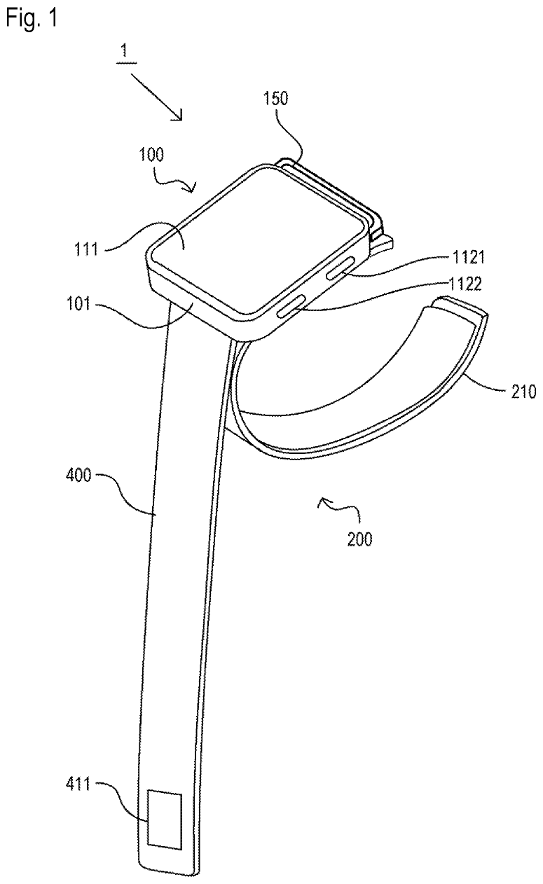

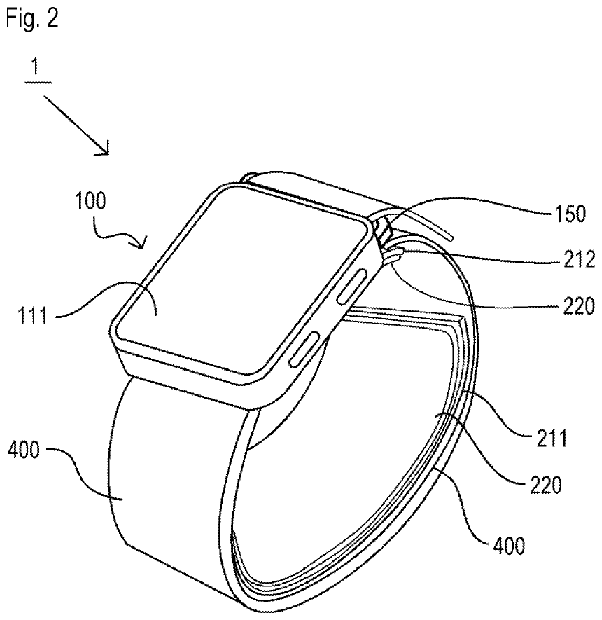

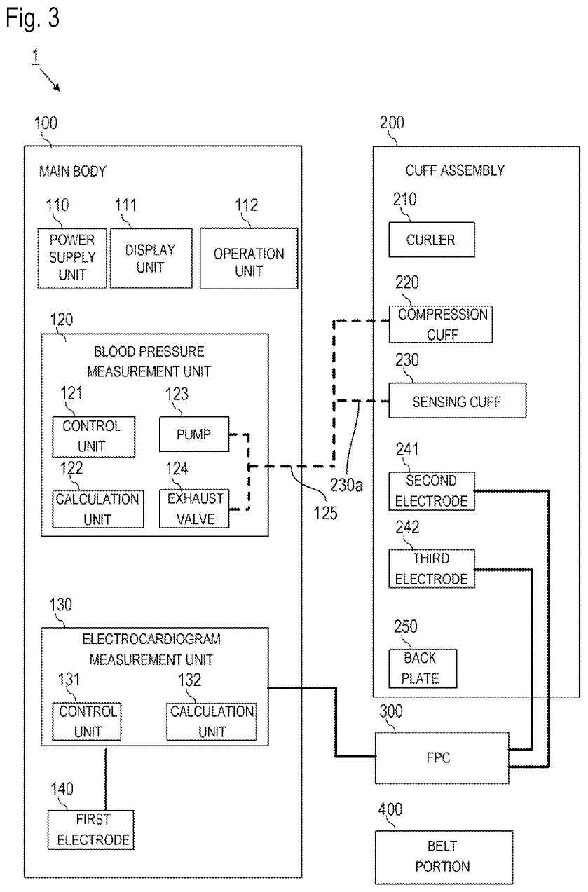

FIGS. 1 and 2 are schematic views illustrating an external configuration of a biological information measurement device 1 according to the present embodiment. FIG. 3 is a functional block diagram illustrating a functional configuration of the biological information measurement device 1 according to the present embodiment.

As illustrated in FIGS. 1 to 3, the biological information measurement device 1 schematically has a configuration which includes a main body 100, a cuff assembly 200, and a belt portion 400, and can measure a blood pressure value and an electrocardiographic waveform in the state of being worn on a wrist T of a subject. The belt portion 400 includes a hook-and-loop fastener 411 including a hook. The main body 100 is provided with a belt passing portion 150 including an annular belt passing ring for inserting the belt portion 400. When the biological information measurement device 1 is worn, the belt portion 400 is wound around the wrist T and then inserted into the belt passing portion 150, and the hook-and-loop fastener 411 is attached to an arbitrary position of the belt portion 400 (a loop with which a hook is engaged is formed) to perform fixing. In addition, the biological information measurement device 1 includes flexible printed circuits (FPCs) 300 (not illustrated in FIGS. 1 and 2) in which wiring for electrically connecting an electrocardiogram measurement unit 130 of the main body 100 to a second electrode 241 and a third electrode 242 of the cuff assembly 200 is arranged. Here, the wrist T corresponds to a measurement target site of the present invention. The biological information measurement device 1 illustrated in FIG. 1 is configured such that the belt-shaped belt portion 400 is wound around the outer circumferential side of the curler 210 formed in a C shape in advance, but the belt portion 400 may be joined and integrated with the outer circumferential side of the C-shaped curler 210. By joining the belt portion 400 with the curler 210 in this manner, the attachment operation of the curler 210 also serves as a winding operation of a part of the belt portion 400, and thus the attachment of the biological information measurement device 1 becomes easy.

As illustrated in FIG. 3, the main body 100 includes a housing 101, a power supply unit 110, a display unit 111, an operation unit 112, a blood pressure measurement unit 120, the electrocardiogram measurement unit 130, and a first electrode 140. Here, the first electrode 140 includes the entire housing 101 of the main body 100 and operation buttons 1121 and 1122. The configuration of the first electrode 140 is not limited thereto, and may be a part of the housing 101 or may be a structure independent of the housing 101. Here, the blood pressure measurement unit 120 corresponds to a blood pressure measurement unit of the present invention, and the electrocardiogram measurement unit 130 corresponds to an electrocardiogram measurement unit and a biological information measurement unit of the present invention. The first electrode 140 corresponds to a first electrode of the present invention.

The power supply unit 110 includes a battery which supplies power necessary for operating the device. For example, the battery may be a secondary battery such as a lithium ion battery, or may be a primary battery.

The display unit 111 includes a display device such as a liquid crystal display, and may include an LED indicator and the like. Specifically, the operation unit 112 includes the operation buttons 1121 and 1122 arranged on the side surface of the housing 101 of the main body 100. A configuration such as a touch panel display may be made in which the display unit 111 and the operation unit 112 are integrated.

The blood pressure measurement unit 120 is a functional unit that controls the cuff assembly 200 to be described later and measures the blood pressure of the user on the basis of information obtained by the cuff assembly, and includes a control unit 121, a calculation unit 122, a pump 123, and an exhaust valve 124. The control unit 121 and the calculation unit 122 are configured by, for example, a central processing unit (CPU) or the like, and may include a storage unit (not illustrated) configured by a random access memory (RAM) or the like. Here, the pump 123 and the exhaust valve 124 correspond to a pump and a valve of the present invention, respectively.

The control unit 121 is a functional unit which controls the blood pressure measurement unit 120, controls the cuff pressure of the cuff assembly 200 via the calculation unit 122, the pump 123, and the like, and acquires information for measuring the blood pressure of the user from the artery on the wrist T on which the biological information measurement device 1 is worn. The calculation unit 122 measures the blood pressure value on the basis of the information acquired in this manner. The pump 123 and the exhaust valve 124 are mechanisms which communicate with a compression cuff 220 and a sensing cuff 230 to be described later via a flow path 125 through which air flows, and are responsible for supplying and discharging air to and from the compression cuff 220 and the sensing cuff 230.

The electrocardiogram measurement unit 130 is a functional unit which measures an electrocardiographic waveform of the user on the basis of a potential difference between the first electrode 140 and second electrode 241 in contact with a human body surface, and includes a control unit 131 and a calculation unit 132. The control unit 131 and the calculation unit 132 are configured by the above-described CPU and the like. From the viewpoint of hardware, the control unit 131 and the calculation unit 132 may have a configuration common to the control unit 121 and the calculation unit 122 of the blood pressure measurement unit 120.

Note that both the blood pressure measurement unit 120 and the electrocardiogram measurement unit 130 include an AD conversion circuit, an amplifier, a filter, and the like (not illustrated) in addition to the CPU, RAM, and the like described above, but since these are configured by known techniques, the description thereof is omitted.

The cuff assembly 200 includes a curler 210, the compression cuff 220, the sensing cuff 230, the second electrode 241, the third electrode 242, and a back plate 250. The curler 210 is a member serving as a base for holding the compression cuff 220. The cuff assembly 200 has a configuration in which the compression cuff 220, the back plate 250, and the sensing cuff 230 are laminated in this order with the curler 210 as the outermost side. Here, the curler 210 corresponds to a support member of the present invention, and the compression cuff 220 (and the sensing cuff 230) corresponds to a fluid bag of the present invention. In addition, the second electrode 241 corresponds to a detection unit and a second electrode of the present invention. In addition, the third electrode 242 corresponds to a third electrode of the present invention.

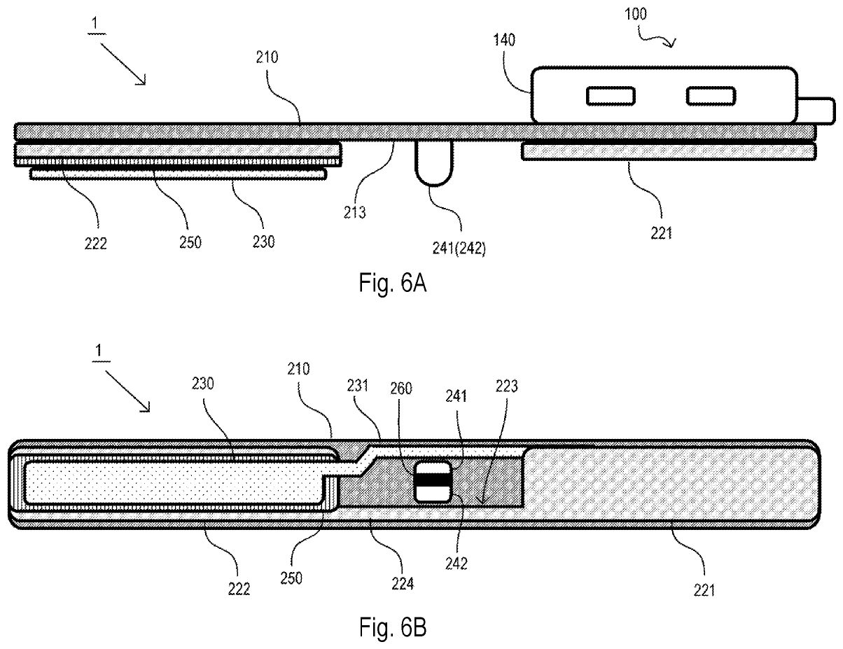

The compression cuff 220 has a role of tightening the wrist T of the wearing portion by being inflated by the air sent from the pump 123 and applying an external pressure to an artery (not illustrated) present on the wrist T. In addition, the sensing cuff 230 (see FIGS. 6A and 6B) is a fluid bag for detecting the pressure applied to the site compressed by the compression cuff 220, and detects, with a small amount of air in the sensing cuff 230, the internal pressure thereof by using a pressure gauge (not illustrated), so as to measure the pressure applied to the compression site. In addition, the back plate 250 (see FIGS. 6A and 6B) is a flexible flat plate member arranged between the compression cuff 220 and the sensing cuff 230, suppresses excessive bending of the sensing cuff 220 at the time of compression by the compression cuff 230, and smooths the pressure distribution in the sensing cuff 230. Here, the air corresponds to a fluid of the present invention.

Both the second electrode 241 and the third electrode 242 are electrodes arranged at positions where the second electrode 241 and the third electrode 242 can be in contact with a human body surface, the second electrode 241 functions as an electrode for electrocardiographic waveform measurement, and the third electrode 242 functions as a ground (GND) electrode for setting a reference potential.

### Structure of Cuff Assembly Portion

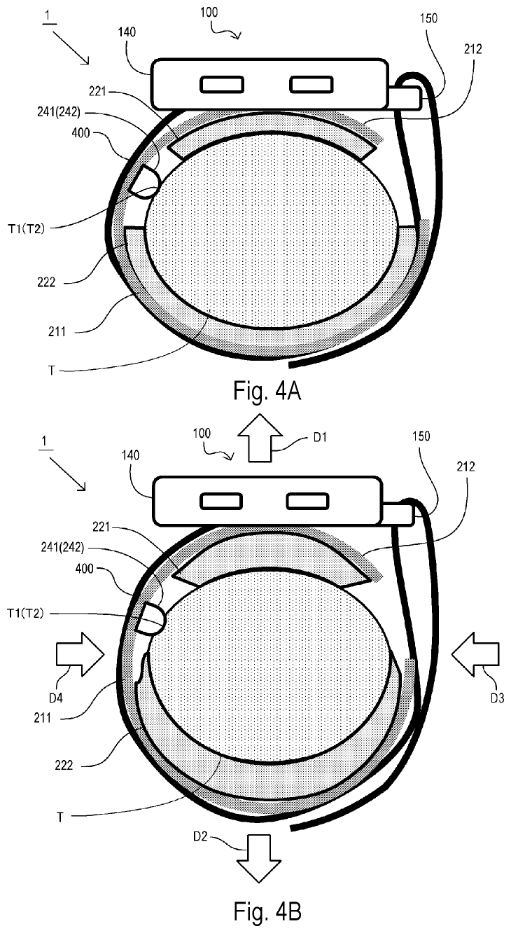

The structure of the cuff assembly 200 will be described on the basis of FIGS. 4A and 4B illustrating a state where the biological information measurement device 1 is worn on the wrist T of the user. In the present embodiment, the compression cuff 220 is provided along the extending direction (a direction around the wrist T) of the curler 210 formed in a C shape curved following the circumferential direction of the wrist T. The curler 210 includes a first curler portion 211 extending in the circumferential direction from the back surface side (the side opposite to the display unit 111) of the main body 100 positioned on the back side of the wrist T, and a second curler portion 212 extending continuously with the first curler portion 211 from the back surface side of the main body 100 to the side opposite to the first curler portion 211 in the circumferential direction. The first curler portion 211 extends so as to cover the artery side of the wrist T.

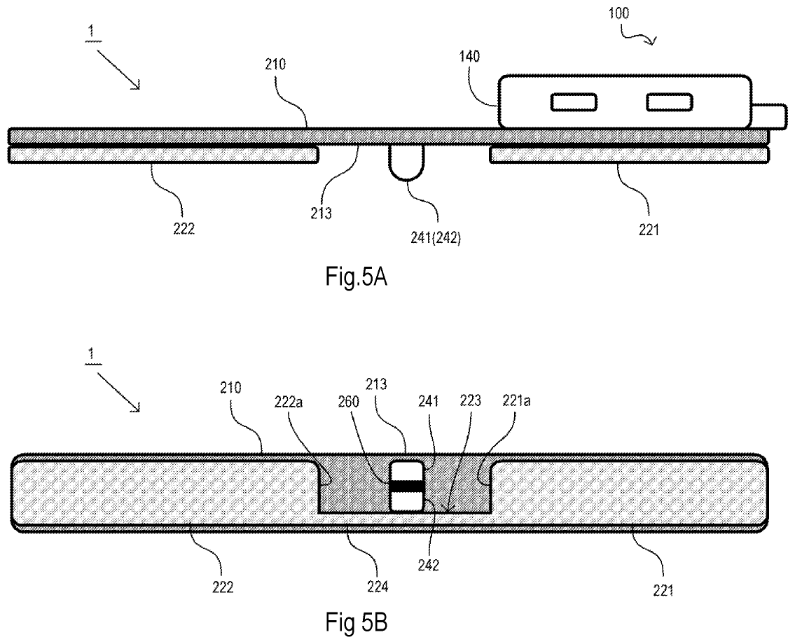

The compression cuff 220 includes a first compression cuff portion 221 and a second compression cuff portion 222 arranged along the circumferential direction. The arrangement of the curler 210, the compression cuff 220, the second electrode 241, and the third electrode 242 will be described with reference to FIGS. 5A and 5B. FIG. 5A is a side view of the biological information measurement device 1 as viewed from a direction orthogonal to the circumferential direction in which the curler 210 is wound. FIG. 5B is a rear view of the biological information measurement device 1 as viewed from the inner diameter side to the outer diameter side in the circumferential direction in which the curler 210 is wound, that is, from the side opposite to the display unit 111 of the main body 100. Note that as illustrated in FIGS. 1, 2, 4A, and 4B, the curler 210 is formed to be curved in a C shape in advance, but FIGS. 5A and 5B illustrate the curler 210 in a state of being developed in the circumferential direction, and do not illustrate the belt portion 400 provided on the outer circumferential side of the curler 210. Here, the first compression cuff portion 221 and the second compression cuff portion 222 correspond to a first bag portion and a second bag portion of the present invention, respectively.

As illustrated in FIG. 5B, the first compression cuff portion 221 and the second compression cuff portion 222 have a cutout portion 223 obtained by cutting out a part of the curler 210 in the width direction (the direction orthogonal to the circumferential direction), and are formed by a series of bag-shaped members communicated by a connection portion 224. The first compression cuff portion 221 and the second compression cuff portion 222 communicate with each other by the connection portion 224 and thus are held at the same pressure, and air is supplied to or discharged from both the first compression cuff portion 221 and the second compression cuff portion 222 under the control of the pump 123 and the exhaust valve 124 described above.

In the curler 210, an exposed portion 213 exposed to the inner circumferential side is formed by the cutout portion 223 of the compression cuff 220. The second electrode 241 and the third electrode 242 are arranged on the inner circumferential side of the exposed portion 213. The second electrode 241 and the third electrode 242 are arranged side by side in the width direction of the exposed portion 213 of the curler 210. The second electrode 241 and the third electrode 242 are made of a conductive member, such as stainless steel, having a substantially semicircular cross section orthogonal to the circumferential direction and a shape, which is obtained by dividing an oval shape into two, in the circumferential direction, and are arranged at predetermined intervals in a direction orthogonal to the circumferential direction. A separator 260 made of an insulating member is arranged between the second electrode 241 and the third electrode 242 (the separator 260 can be omitted). Here, the exposed portion 213 of the curler 210 corresponds to a detection unit support portion and an electrode support portion of the present invention.

The second electrode 241 and the third electrode 242 are provided at positions separated by a predetermined distance from a circumferential end portion 221a of the first compression cuff portion 221 on the cutout portion 223 side and a circumferential end portion 222a of the second compression cuff portion 222 on the cutout portion 223 side, respectively. The predetermined distance from each of the circumferential end portion 221a of the first compression cuff portion 221 on the cutout portion 223 side and the circumferential end portion 222a of the second compression cuff portion 222 on the cutout portion 223 side is a distance that can limit the movement and posture change of the second electrode 241 and the third electrode 242 to a predetermined range when air is supplied to the compression cuff 220 to inflate the compression cuff 220 as described later. By arranging the second electrode 241 and the third electrode 242 at such positions of the exposed portion 213 of the curler 210, a change in the contact state of the second electrode 241 and the third electrode 242 with the wrist T is suppressed even when the compression cuff 220 is inflated. Here, the change in the contact state is a change in the contact state such as a contact position, a contact angle, and a contact area of the second electrode 241 and the third electrode 242 with the wrist T. As described above, in the biological information measurement device 1, the second electrode 241 and the third electrode 242 are arranged on the inner circumferential side of the curler 210 between the first compression cuff portion 221 and the second compression cuff portion 222 arranged along the circumferential direction. Here, the circumferential end portion 221a of the first compression cuff portion 221 on the cutout portion 223 side corresponds to a first end portion, on the detection unit side, of the first bag portion of the present invention, and the predetermined distance by which the circumferential end portion 221a of the first compression cuff portion 221 on the cutout portion 223 side is separated from the second electrode 241 and the third electrode 242 corresponds to a first predetermined distance of the present invention. In addition, the circumferential end portion 222a of the second compression cuff portion 222 on the cutout portion 223 side corresponds to a first end portion, on the detection unit side, of the second bag portion of the present invention, and the predetermined distance by which the circumferential end portion 222a of the second compression cuff portion 222 on the cutout portion 223 side is separated from the second electrode 241 and the third electrode 242 corresponds to a second predetermined distance of the present invention.

FIGS. 6A and 6B are a side view and a rear view illustrating the biological information measurement device 1 having the above-described configuration, the side view and the back view including the sensing cuff 230 and the back plate 250 arranged on the inner circumferential side of the compression cuff 220. A configuration is made in which the second compression cuff portion 222, the back plate 250, and the sensing cuff 230 are laminated in this order with the curler 210 as the outermost side. The sensing cuff 230 is connected to a flow path 230a connected to the flow path 125 which communicates the pump 123 and the exhaust valve 124 with the compression cuff 220, and air is supplied and discharged. The flow path 230a is arranged on the inner circumferential side of the exposed portion 213 of the curler 210 such that the second electrode 241 and the third electrode 242 are sandwiched between the flow path 230a and the connection portion 224, and extends toward the main body 100. The sensing cuff 230 and the back plate 250 are set to a size that does not allow interference with the second electrode 241 and the third electrode 242 beyond the circumferential end portion 222a of the second compression cuff portion 222 on the cutout portion 223 side even when the compression cuff 220 and the sensing cuff 230 are inflated when the biological information measurement device 1 is worn.

Note that although not illustrated, a cover which covers the inner circumferential side of each of the first compression cuff portion 221, the second compression cuff portion 222, the sensing cuff 230, and the back plate 250 is provided.

Returning to FIGS. 4A and 4B, the operation of the biological information measurement device 1 worn on the wrist T of the user will be described.

FIG. 4A illustrates a state where the belt portion 400 is wound around the wrist T on the outer circumferential side of the curler 210, is inserted into the belt passing portion 150, and fixed by the hook-and-loop fastener 411, and the compression cuff 220 is not inflated, and FIG. 4B illustrates a state after the compression cuff 220 is inflated. As described above, when the compression cuff 220 arranged on the inner circumferential side is inflated in a state where the circumferential length is restricted by the belt portion 400 wound around the wrist T, the first compression cuff portion 221 covering the artery side of the wrist T and the second compression cuff portion 222 covering the back side of the wrist T are both inflated, whereby the C-shaped curler 210 expands in the back direction of the wrist T indicated by an arrow D1 and the artery direction indicated by an arrow D2, and is deformed to narrow toward the inner circumferential side in the width direction of the wrist T orthogonal to the arrow D1 and the arrow D2 as indicated by an arrow D3 and an arrow D4. Since the curler 210 is deformed in this manner by the inflation of the compression cuff 220, a pressing force from the curler 210 toward the wrist T acts on the second electrode 241 and the third electrode 242 arranged on the exposed portion 213 exposed to the wrist T side by the cutout portion 223 between the first compression cuff portion 221 and the second compression cuff portion 222 in the circumferential direction. Therefore, even when the compression cuff 220 is inflated, it is possible to maintain the stable contact of the second electrode 241 and the third electrode 242 with the wrist T, and it is possible to suppress a change in the contact state of the second electrode 241 and the third electrode 242 with the wrist T. In addition, the blood pressure measurement is not hindered by the measurement of the electrocardiographic waveform.

Here, the inflation of the compression cuff 220 corresponds to a volume change of the fluid bag of the present invention.

In the shapes of the second electrode 241 and the third electrode 242, a cross-sectional shape in the direction orthogonal to the circumferential direction is not limited to a semicircular shape as illustrated in FIGS. 4A and 4B, and may be a semi-elliptical shape, a semi-oval shape, or another curved shape such as a curve protruding toward the wrist T.

### Measurement of Biological Information

In order to measure the biological information by the biological information measurement device 1 having the above configuration, first, the cuff assembly 200 and the belt portion 400 are wound around the wrist T such that the main body 100 faces the back side of the hand. Then, the belt portion 400 is passed through the belt passing portion 150 and folded back, the hook-and-loop fastener 411 of the belt portion 400 is stuck to an arbitrary position of the belt portion 400, and the biological information measurement device 1 is worn on the wrist T. At this time, the sensing cuff 230 is worn to be positioned on the palm side of the wrist T.

Then, the measurement is started by operating the operation button 1121 (or 1122). Specifically, the compression cuff 220 is inflated by injecting air into the compression cuff 220, so as to compress (the artery of) the wrist T, obstruct the artery, and temporarily stop blood flow, then the compression cuff 220 is contracted by gradually discharging the air from the compression cuff 220, so as to release the compression and restore the blood flow in the artery, and the pressure at that time is measured by the sensing cuff 230. That is, the blood pressure measurement by a so-called oscillometric method is performed.

Then, during the blood pressure measurement, when the wrist T is compressed by the compression cuff 220, the second electrode 241 and the third electrode 242 come into contact with (are pressed against) surfaces T1 and T2 (see FIG. 5A) of the wrist T. Therefore, when the first electrode 140 provided in the housing 101 of the main body 100 is touched with the finger on which the biological information measurement device 1 is not worn, the electrocardiographic waveform can be measured by a so-called I induction method on the basis of the potential difference between the first electrode 140 and the second electrode 241. Here, the finger on which the biological information measurement device 1 is not worn corresponds to a first site of the present invention, and the surfaces T1 and T2 of the wrist T correspond to a second site and a third site of the present invention, respectively.

As described above, according to the biological information measurement device 1 of the present embodiment, the blood pressure value and the electrocardiographic waveform can be simultaneously and accurately measured by a portable device of the type worn on the wrist T with a configuration easy to manufacture.

### First Modification

As the biological information measurement device 1 according to the first embodiment, the device which measures the electrocardiographic waveform as the biological information together with the blood pressure has been described, but the biological information is not limited thereto. When a photoplethysmography sensor (PPG sensor) is arranged instead of the second electrode 241 and the third electrode 242 in the exposed portion 213 of the curler 210 in which the second electrode 241 and the third electrode 242 described above are arranged, it is possible to constitute a biological information measurement device which measures, as biological information, a pulse wave or information based on the pulse wave together with blood pressure measurement. According to such a biological information measurement device, it is possible to suppress a change in the contact state of the PPG sensor with the measurement target site due to the inflation of the compression cuff 220, and the blood pressure measurement is not hindered by the measurement of the electrocardiographic waveform. The blood pressure value and the pulse wave can be simultaneously and accurately measured by a portable device of the type worn on the wrist T with a configuration easy to manufacture. Here, the photoplethysmography sensor corresponds to a detection unit of the present invention, and the pulse wave or the information based on the pulse wave corresponds to biological information of the present invention.

In addition, when an arterial oxygen saturation sensor (SpO2 sensor) is arranged in the exposed portion 213 of the curler 210 in which the second electrode 241 and the third electrode 242 described above are arranged, it is possible to constitute a biological information measurement device which measures, as biological information, arterial oxygen saturation or information based on the arterial oxygen saturation together with blood pressure measurement. According to such a biological information measurement device, it is possible to suppress a change in the contact state of the SpO2 sensor with the measurement target site due to the inflation of the compression cuff 220, and the blood pressure measurement is not hindered by the measurement of the arterial oxygen saturation. The blood pressure value and the arterial oxygen saturation can be simultaneously and accurately measured by a portable device of the type worn on the wrist T with a configuration easy to manufacture. Here, the arterial oxygen saturation sensor corresponds to a detection unit of the present invention, and the arterial oxygen saturation or the information based on the arterial oxygen saturation corresponds to biological information of the present invention.

### Second Modification

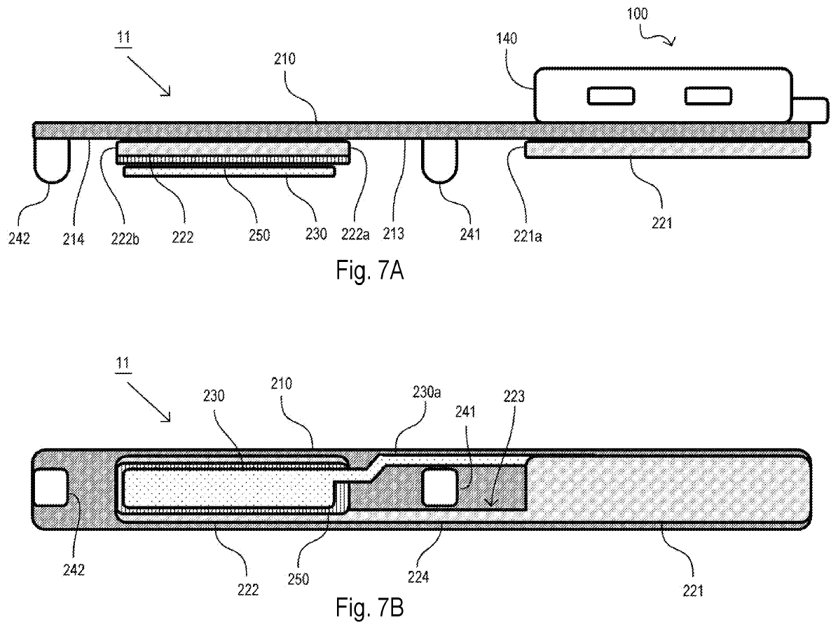

FIGS. 7A and 7B illustrate a biological information measurement device 11 according to a modification of the biological information measurement device 1 according to the first embodiment. The same configurations as those of the biological information measurement device 1 according to the first embodiment are denoted by the same reference numerals, and detailed description thereof will be omitted.

In the biological information measurement device 11, the second electrode 241 and the third electrode 242 are arranged at different positions in the circumferential direction with respect to the curler 210. Similarly to the biological information measurement device 1, the second electrode 241 is arranged in the exposed portion 213 of the curler 210 formed by the cutout portion 223 provided between the first compression cuff portion 221 and the second compression cuff portion 222. Also in the biological information measurement device 11, the second electrode 241 is provided at a position separated by a predetermined distance from each of the circumferential end portion 221a of the first compression cuff portion 221 on the cutout portion 223 side and the circumferential end portion 222a of the second compression cuff portion 222 on the cutout portion 223 side.

In the biological information measurement device 11, the curler 210 has an extension portion 214 further extending from a circumferential end portion 222b on the side opposite to the main body 100, that is, on the side opposite to the circumferential end portion 222a of the second compression cuff portion 222. The compression cuff 220 and the like are not arranged on the inner circumferential side of the extension portion 214, and the extension portion 214 of the curler 210 is exposed to the inner circumferential side. Then, in the biological information measurement device 11, the third electrode 242 is arranged on the inner circumferential side of the extension portion 214. The third electrode 242 is arranged apart from the circumferential end portion 222b of the second compression cuff portion 222 by a predetermined distance. The predetermined distance from the circumferential end portion 222b of the second compression cuff portion 222 is a distance which can limit the movement and posture change of the third electrode 242 to a predetermined range when air is supplied to the compression cuff 220 to inflate the compression cuff 220. By arranging the third electrode 242 at such a position of the extension portion 214 of the curler 210, a change in the contact state between the third electrode 242 and the wrist T is suppressed even when the compression cuff 220 is inflated. In addition, the blood pressure measurement is not hindered by the measurement of the electrocardiographic waveform. Here, the circumferential end portion 222b of the second compression cuff portion 222 corresponds to a second end portion of the second bag portion of the present invention, and the extension portion 214 corresponds to an extension portion of the present invention. In addition, a predetermined distance by which the third electrode 242 is separated from the circumferential end portion 222b of the second compression cuff portion 222 corresponds to a third predetermined distance of the present invention.

In the biological information measurement device 11, the third electrode 242 is arranged in the extension portion 214 which is the end portion of the curler 210 in the circumferential direction. However, as illustrated in FIGS. 4A and 4B, in a state where the biological information measurement device 11 is worn on the wrist T of the user, the belt portion 400 is wound around the outer circumferential side of the curler 210, and thus, a force of applying pressure to the inner diameter side toward the wrist T also acts on the third electrode 242.

As described above, even when the second electrode 241 and the third electrode 242 are arranged at different positions of the curler 210 in the circumferential direction, the blood pressure value and the electrocardiographic waveform can be simultaneously and accurately measured by a portable device of the type worn on the wrist T with a configuration easy to manufacture.

### Third Modification

In the first embodiment described above, the configuration in which the sensing cuff 230 and the back plate 250 are provided to perform the blood pressure measurement by the oscillometric method has been described as the biological information measurement device 1, but the blood pressure measurement method is not limited thereto, and a configuration may be made in which the blood pressure measurement may be performed by detecting a Korotkoff sound with a microphone or the like.

In a case where the blood pressure measurement is performed by such a method, as illustrated in FIGS. 5A and 5B, the biological information measurement device 1 includes the compression cuff 220, the second electrode 241, and the third electrode 242, but does not include the sensing cuff 230 and the back plate 250.

Also in the biological information measurement device 1 according to the third modification, similarly, even when the compression cuff 220 is inflated, it is possible to maintain the stable contact of the second electrode 241 and the third electrode 242 with the wrist T, and it is possible to suppress a change in the contact state of the second electrode 241 and the third electrode 242 with the wrist T. In addition, the blood pressure measurement is not hindered by the measurement of the electrocardiographic waveform, and the biological information measurement device 1 according to the third modification can simultaneously and accurately measure the blood pressure value and the electrocardiographic waveform with a configuration easy to manufacture.

### Second Embodiment

Hereinafter, a biological information measurement device 12 according to a second embodiment of the present invention will be described with reference to the drawings. The same configurations as those of the biological information measurement device 1 according to the first embodiment are denoted by the same reference numerals, and detailed description thereof will be omitted.

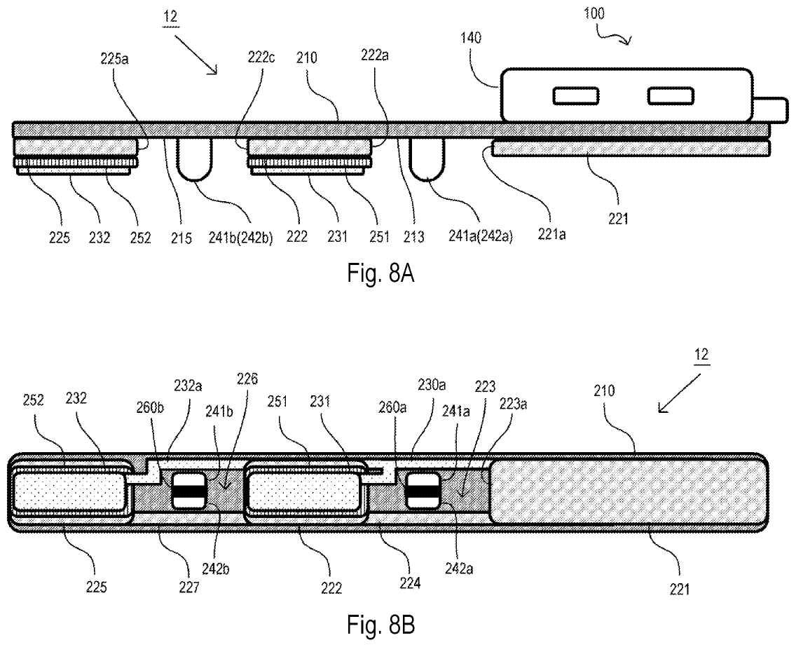

FIG. 8A is a side view in which the curler 210 of the biological information measurement device 12 is developed in the circumferential direction, and FIG. 8B is a rear view of the biological information measurement device 12. In FIGS. 8A and 8B, illustration of the belt portion 400 and the cover is omitted as in FIG. 5 and the like.

In the biological information measurement device 12, the compression cuff 220 includes a third compression cuff portion 225 in addition to the first compression cuff portion 221 and the second compression cuff portion 222. In addition to the cutout portion 223 obtained by cutting out a part in the width direction between the first compression cuff portion 221 and the second compression cuff portion 222 in the circumferential direction, the compression cuff 220 includes a cutout portion 226 obtained by cutting out a position in the width direction between the second compression cuff portion 222 and the third compression cuff portion 225 in the circumferential direction. The second compression cuff portion 222 and the third compression cuff portion 225 are formed of a series of bag-shaped members communicated by the connection portion 227. Therefore, the first compression cuff portion 221, the second compression cuff portion 222, and the third compression cuff portion 225 are configured as a series of bag-shaped members communicated by the connection portion 224 and the connection portion 227, and thus are held at the same pressure, and air is supplied to or discharged from all of the first compression cuff portion 221, the second compression cuff portion 222, and the third compression cuff portion 225 under the control of the pump 123 and the exhaust valve 124 described above. Here, the third compression cuff portion 225 corresponds to a third bag portion of the present invention.

In the curler 210, similarly to the exposed portion 213, an exposed portion 215 is formed by the cutout portion 226 of the compression cuff 220. In the exposed portion 215, a second electrode (2) 241b and a third electrode (2) 242b are arranged side by side in the width direction. In order to distinguish from these, in the present embodiment, the electrodes arranged in the exposed portion 213 between the first compression cuff portion 221 and the second compression cuff portion 222 is referred to as a second electrode (1) 241a and a third electrode (1) 242a, and the separator arranged therebetween is referred to as a separator 260a. The second electrode (2) 241b and the third electrode (2) 242b are in contact with the sites of surfaces different from the surfaces T1 and T2 of the wrist T with which the second electrode (1) 241a and the third electrode (1) 242a are in contact, respectively. The second electrode (2) 241b and the third electrode (2) 242b are made of a conductive member, such as stainless steel, having a substantially semicircular cross section orthogonal to the circumferential direction and a shape, which is obtained by dividing an oval shape into two, in the circumferential direction, and are arranged at predetermined intervals in a direction orthogonal to the circumferential direction. A separator 260b made of an insulating member is arranged between the second electrode (1) 241a and the third electrode (2) 242b (the separator 260b can be omitted). Here, the exposed portion 215 corresponds to a second electrode support portion of the present invention. In addition, the second electrode (2) 241b and the third electrode (2) 242b correspond to a fourth electrode and a fifth electrode of the present invention, respectively, and portions on the surface of the wrist T of the user with which the second electrode (2) 241b and the third electrode (2) 242b are in contact correspond to a fourth site and a fifth site of the present invention, respectively.

In the shapes of the second electrode (1) 241a, the second electrode (2) 241b, the third electrode (1) 242a, and the third electrode (2) 242b, a cross-sectional shape in the direction orthogonal to the circumferential direction is not limited to a semicircular shape as illustrated in FIG. 8, and may be a semi-elliptical shape, a semi-oval shape, or another curved shape such as a curve protruding toward the wrist T.

The second electrode (2) 241b and the third electrode (2) 242b are provided at positions separated by a predetermined distance from a circumferential end portion 222c of the second compression cuff portion 222 on the cutout portion 226 side and a circumferential end portion 225a of the third compression cuff portion 225 on the cutout portion 226 side, respectively. The predetermined distance from each of the circumferential end portion 222c of the second compression cuff portion 222 on the cutout portion 226 side and the circumferential end portion 225a of the third compression cuff portion 225 on the cutout 226 side is a distance that can limit the movement and posture change of the second electrode (2) 241b and the third electrode (2) 242b to a predetermined range when air is supplied to the compression cuff 220 to inflate the compression cuff 220. By arranging the second electrode (2) 241b and the third electrode (2) 242b at such positions of the exposed portion 215 of the curler 210, a change in the contact state of the second electrode (2) 241b and the third electrode (2) 242b with the wrist T is suppressed even when the compression cuff 220 is inflated. Here, the change in the contact state is a change in the contact state such as a contact position, a contact angle, and a contact area of the second electrode (2) 241b and the third electrode (2) 242b with the wrist T. As described above, in the biological information measurement device 12, on the inner circumferential side of the curler 210, the second electrode (1) 241a and the third electrode (1) 242a are arranged between the first compression cuff portion 221 and the second compression cuff portion 222 arranged along the circumferential direction, and the second electrode (2) 241b and the third electrode (2) 242b are arranged between the second compression cuff portion 222 and the third compression cuff portion 225 similarly arranged along the circumferential direction. Since the curler 210 is deformed as illustrated in FIG. 4B by the inflation of the compression cuff 220, a pressing force from the curler 210 toward the wrist T acts on the second electrode (1) 241a and the third electrode (1) 242a, which are arranged in the exposed portion 213 exposed to the wrist T side, by the cutout portion 223 between the first compression cuff portion 221 and the second compression cuff portion 222 in the circumferential direction and the second electrode (2) 241b and the third electrode (2) 242b, which are arranged in the exposed portion 215 exposed to the wrist T side, by the cutout portion 226 between the second compression cuff portion 222 and the third compression cuff portion 225 in the circumferential direction. Therefore, even when the compression cuff 220 is inflated, it is possible to maintain the stable contact of the second electrode (1) 241a, the second electrode (2) 241b, the third electrode (1) 242a, and the third electrode (2) 242b with the wrist T, and it is possible to suppress a change in the contact state of the second electrode (1) 241a, the second electrode (2) 241b, the third electrode (1) 242a, and the third electrode (2) 242b with the wrist T. In addition, the blood pressure measurement is not hindered by the measurement of the electrocardiographic waveform. Here, the circumferential end portion 222c of the second compression cuff portion 222 on the cutout portion 226 side corresponds to a second end portion, on the fourth electrode side, of the second bag portion of the present invention, and the circumferential end portion 225a of the third compression cuff portion 225 on the cutout portion 226 side corresponds to a first end portion, on the fourth electrode side, of the third bag portion of the present invention. In addition, the predetermined distances by which the second electrode (2) 241b is separated from the circumferential end portion 222c of the second compression cuff portion 222 on the cutout portion 226 side and the circumferential end portion 225a of the third compression cuff portion 225 on the cutout portion 226 side correspond to a fourth predetermined distance and a fifth predetermined distance of the present invention, respectively.

Similarly to the biological information measurement device 1 according to the first embodiment, on the inner circumferential side of the second compression cuff portion 222, the second compression cuff portion 222, a back plate 251, and a first sensing cuff portion 231 are sequentially laminated with the curler 210 as the outermost side. In addition, on the inner circumferential side of the third compression cuff portion 225, the third compression cuff portion 225, a back plate 252, and a second sensing cuff portion 232 are sequentially laminated with the curler 210 as the outermost side. The first sensing cuff portion 231 and the second sensing cuff portion 232 are configured as a series of bag-shaped members communicating with each other by a connection portion 232a, and communicate with the flow path 230a. Here, the first sensing cuff portion 231 and the second sensing cuff portion 232 together function as the sensing cuff 230. The first sensing cuff portion 231 and the back plate 251 are set to a size that does not allow interference with the second electrode (1) 241a and the third electrode (1) 242a beyond the circumferential end portion 222a of the second compression cuff portion 222 on the cutout portion 223 side and does not allow interference with the second electrode (2) 241b and the third electrode (2) 242b beyond the circumferential end portion 222c of the second compression cuff portion 222 on the cutout portion 226 side even when the second compression cuff portion 222 and the first sensing cuff portion 231 is inflated when the biological information measurement device 12 is worn. Similarly, the second sensing cuff portion 232 and the back plate 252 are set to a size that does not allow interference with the second electrode (2) 241b and the third electrode (2) 242b beyond the circumferential end portion 225a of the third compression cuff portion 225 on the cutout portion 226 side even when the third compression cuff portion 225 and the second sensing cuff portion 232 are inflated when the biological information measurement device 12 is worn.

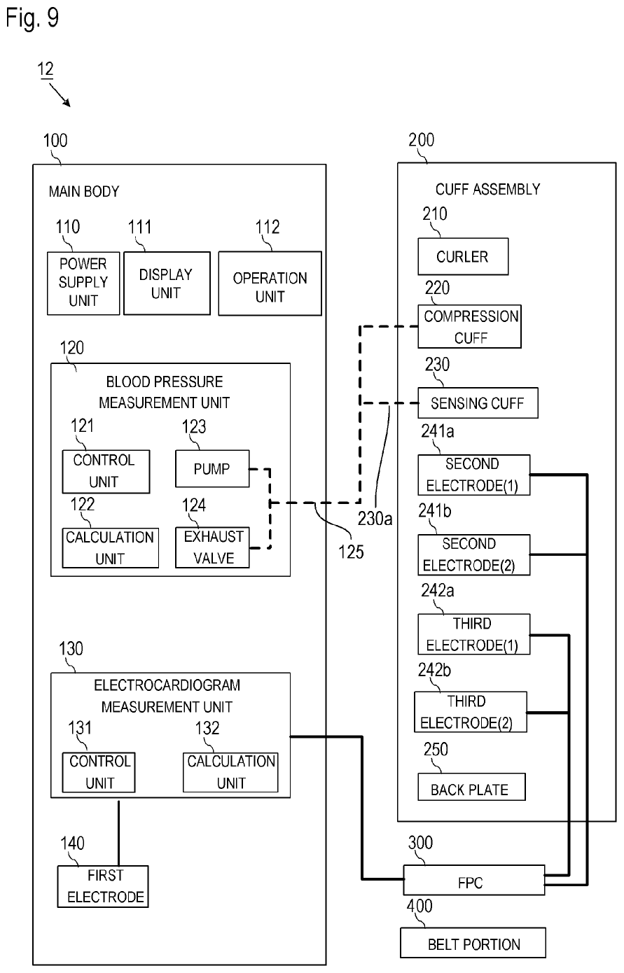

FIG. 9 is a functional block diagram illustrating a functional configuration of the biological information measurement device 12. The description of the same configuration as that of the biological information measurement device 1 will be omitted. Here, the second electrode (1) 241a and the second electrode (2) 241b are electrically connected to each other, and perform input to the electrocardiogram measurement unit 130 via the FPC 300. The third electrode (1) 242a and the third electrode (2) 242b are electrically connected to each other. Therefore, the measurement of the electrocardiographic waveform using two of the second electrodes (1) 241a and the second electrodes (2) 241b and two of the third electrodes (1) 242a and the third electrodes (2) 242b is performed similarly to that in the biological information measurement device 1 according to the first embodiment. Since the second electrode (1) 241a and the second electrode (2) 241b are electrically connected to each other, the contact area of the second electrode is substantially widened, and thus the electrocardiographic waveform can be more stably measured.

As described above, the blood pressure value and the electrocardiographic waveform can be simultaneously and accurately measured by a portable device of the type worn on the wrist T with a configuration easy to manufacture.

### Modification

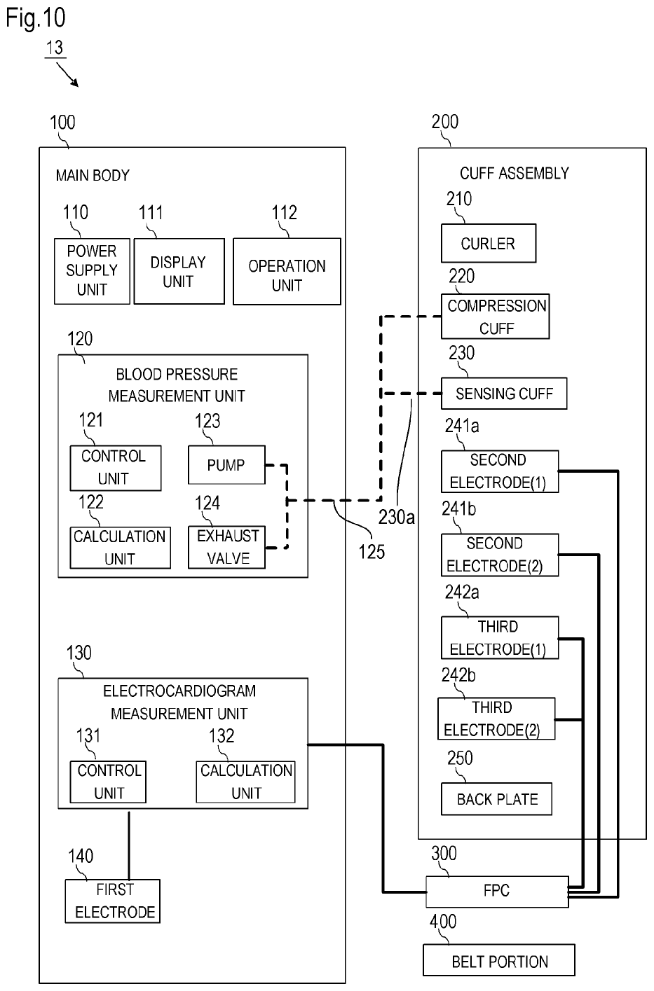

FIG. 10 is a functional block diagram illustrating a functional configuration of the biological information measurement device 13 according to a modification of the biological information measurement device 12. The configuration of the biological information measurement device 13 is the same as that of the biological information measurement device 12 illustrated in FIG. 7, but only the connection method between the second electrode (1) 241a and the second electrode (2) 241b is different. In the biological information measurement device 13, the second electrode (1) 241a and the second electrode (2) 241b are not connected, and independently perform input to the electrocardiogram measurement unit 130 via the FPC 300. Note that the third electrode (1) 242a and the third electrode (2) 242b are electrically connected to each other. Therefore, in the biological information measurement device 13, the electrocardiogram measurement unit 130 can acquire electrocardiographic waveform (1) recorded from the potential difference between first electrode 140 and second electrode (1) 241a and electrocardiographic waveform (2) recorded from the potential difference between first electrode 140 and second electrode (2) 241b, so that it is possible to select and store the electrocardiographic waveform having better waveform quality or the electrocardiographic waveform having lower contact resistance, thereby enabling more accurate electrocardiographic waveform measurement. Here, the potential difference between the first electrode 140 and the second electrode (1) 241a corresponds to a first potential difference of the present invention, and the potential difference between the first electrode 140 and the second electrode (2) 241b corresponds to a second potential difference of the present invention.

### Third Embodiment

In the first and second embodiments, the first compression cuff portion 221, the second compression cuff portion 222 or the third compression cuff portion 225, the second electrode 241 (241a, 241b), the third electrode 242 (242a, 242b), and the like are arranged on the inner circumferential side of the curler 210 provided independently of the belt portion 400, but the curler 210 may be omitted, and each member arranged on the inner circumferential side of the curler 210 may be arranged on the inner circumferential side of the belt portion 400.

At this time, in the configuration corresponding to the first embodiment, the first compression cuff portion 221, the second compression cuff portion 222, the second electrode 241, and the third electrode 242 are arranged on the inner circumferential side of the belt portion 400, or the first compression cuff portion 221, the second compression cuff portion 222, the back plate 250, the sensing cuff 230, the second electrode 241, and the third electrode 242 are arranged on the inner circumferential side of the belt portion 400. The relative positional relationship of the members arranged on the inner circumferential side of the belt portion 400 is similar to that of the biological information measurement device 1 of the first embodiment arranged on the inner circumferential side of the curler 210.

In the configuration corresponding to the first modification of the first embodiment, a PPG sensor or a SpO2 sensor is arranged instead of the second electrode 241 and the third electrode 242 on the inner circumferential side of the belt portion 400.

In addition, the configuration corresponding to the second modification of the first embodiment is the same as the configuration corresponding to the first embodiment described above except that the second electrode 241 and the third electrode 242 are arranged at different positions of the belt portion 400 in the circumferential direction.

In addition, in the configuration corresponding to the third modification of the first embodiment, as in Example 1, the first compression cuff portion 221, the second compression cuff portion 222, the second electrode 241, and the third electrode 242 are arranged on the inner circumferential side of the belt portion 400, and a microphone or the like for detecting Korotkoff sound is further arranged.

In the configuration corresponding to the second embodiment, the first compression cuff portion 221, the second compression cuff portion 222, the third compression cuff portion 225, the second electrode (1) 241a, the third electrode (1) 242a, the second electrode (2) 241b, and the third electrode (2) 242b are arranged on the inner circumferential side of the belt portion 400, or the first compression cuff portion 221, the second compression cuff portion 222, the back plate 251, the first sensing cuff portion 231, the third compression cuff portion 225, the back plate 252, the second sensing cuff portion 232, the second electrode (1) 241a, the third electrode (1) 242a, the second electrode (2) 241b, and the third electrode (2) 242b are arranged on the inner circumferential side of the belt portion 400. The relative positional relationship of the members arranged on the inner circumferential side of the belt portion 400 is similar to that of the biological information measurement device 1 of the second embodiment arranged on the inner circumferential side of the curler 210.

As described above, when the curler 210 is omitted and the first compression cuff portion 221, the second compression cuff portion 222, the second electrode 241 (241a, 241b), the third electrode 242 (242a, 242b), the PPG sensor, the SpO2 sensor, or the like is arranged on the inner circumferential side of the belt portion 400, the configuration of the biological information measurement device can be simplified, and handling is also facilitated.

### REFERENCE SIGNS LIST

1, 11, 12, 13 biological information measurement device
120 blood pressure measurement unit
123 pump
124 exhaust valve
130 electrocardiogram measurement unit
210 curler
220 compression cuff
221 first compression cuff portion
222 second compression cuff portion
241 second electrode

1. A biological information measurement device which measures blood pressure of a subject and biological information different from the blood pressure, the biological information measurement device comprising:
a belt portion which is wound around an outer circumference of a measurement target site of the subject and fixes the biological information measurement device to the measurement target site;
a fluid bag which is arranged on an inner circumferential side of a support member;
a pump which supplies a fluid into the fluid bag;
a valve which is provided in a flow path of the fluid communicating with the fluid bag;
a blood pressure measurement unit which compresses the measurement target site by supplying the fluid from the pump to inflate the fluid bag or releases compression on the measurement target site by controlling the valve to discharge the fluid in the fluid bag to contract the fluid bag, and measures a blood pressure of the measurement target site;
a detection unit which comes into contact with the measurement target site of the subject and is configured to measure the biological information; and
a biological information measurement unit which measures the biological information by using the detection unit,
wherein the fluid bag includes a first bag portion and a second bag portion which are arranged apart from each other in a circumferential direction of the support member and communicate with each other, and
the detection unit
is supported by a detection unit support portion which is the support member exposed to an inner circumferential side between the first bag portion and the second bag portion, and
is arranged at a position which allows a change in a contact state between the detection unit and the measurement target site due to movement of the detection unit support portion accompanying a volume change of the first bag portion and the second bag portion to be limited to a predetermined range, the position being a position separated by a first predetermined distance from a first end portion of the first bag portion on the detection unit side and separated by a second predetermined distance from a first end portion of the second bag portion on the detection unit side.

2. The biological information measurement device according to claim 1, wherein the detection unit is a photoplethysmography sensor, and the biological information is a pulse wave or information based on the pulse wave.

3. The biological information measurement device according to claim 1, wherein the detection unit is an arterial oxygen saturation sensor, and the biological information is arterial oxygen saturation or information based on the arterial oxygen saturation.

4. The biological information measurement device according to claim 1, wherein the biological information is an electrocardiographic waveform, the device further comprising:
a first electrode which comes into contact with a first site of the subject;
a second electrode which is the detection unit and comes into contact with a second site which is the measurement target site different from the first site;
an electrode support portion which is the detection unit support portion; and
an electrocardiogram measurement unit which is the biological information measurement unit and measures the electrocardiographic waveform of the subject through the first electrode and the second electrode.

5. The biological information measurement device according to claim 4, further comprising a third electrode which comes into contact with a third site of the subject different from the first site and the second site and sets a reference potential,
wherein the third electrode
is supported by an extension portion of the support member extending, to a side opposite to the first end portion, from a second end portion of the second bag portion on a side opposite to the first end portion, and
is arranged at a position which allows a change in a contact state between the third electrode and the third site due to movement of the extension portion accompanying a volume change of the second bag portion to be limited to a predetermined range, the position being a position separated by a third predetermined distance from the second end portion of the second bag portion.

6. The biological information measurement device according to claim 4, further comprising a third electrode which comes into contact with a third site of the subject different from the first site and the second site and sets a reference potential,
wherein the third electrode is supported by the electrode support portion, and is arranged at a same position as the second electrode in the circumferential direction with respect to the first bag portion and the second bag portion and at a position aligned with the second electrode in a direction orthogonal to the circumferential direction.

7. The biological information measurement device according to claim 4, further comprising a fourth electrode which comes into contact with a fourth site of the subject different from the first site and the second site,
wherein the electrocardiogram measurement unit measures the electrocardiographic waveform of the subject through the first electrode, the second electrode, and the fourth electrode,
the fluid bag further includes a third bag portion which is arranged apart from the second bag portion on a side opposite to the first bag portion in the circumferential direction of the support member and communicates with the second bag portion, and
the fourth electrode is supported by a second electrode support portion which is the support member exposed to an inner circumferential side between the second bag portion and the third bag portion, and is arranged at a position which allows a change in a contact state between the fourth electrode and the fourth site due to movement of the second electrode support portion accompanying a volume change of the second bag portion and the third bag portion to be limited to a predetermined range, the position being a position separated by a fourth predetermined distance from a second end portion of the second bag portion on the fourth electrode side and separated by a fifth predetermined distance from a first end portion of the third bag portion on the fourth electrode side.

8. The biological information measurement device according to claim 7, wherein the fourth electrode is electrically connected to the second electrode.

9. The biological information measurement device according to claim 7, wherein
the fourth electrode is not electrically connected to the second electrode, and
the electrocardiogram measurement unit measures the electrocardiographic waveform on a basis of any one of a first potential difference detected by the first electrode and the second electrode or a second potential difference detected by the first electrode and the fourth electrode.

10. The biological information measurement device according to claim 7, further comprising:
a third electrode which comes into contact with a third site of the subject different from any one of the first site, the second site, and the fourth site and sets a reference potential; and
a fifth electrode which comes into contact with a fifth site of the subject different from any one of the first site, the second site, the third site, and the fourth site and is electrically connected to the third electrode,
wherein the third electrode is supported by the electrode support portion, and is arranged at a same position as the second electrode in the circumferential direction with respect to the first bag portion and the second bag portion and at a position aligned with the second electrode in a direction orthogonal to the circumferential direction, and
the fifth electrode is supported by the second electrode support portion, and is arranged at a same position as the fourth electrode in the circumferential direction with respect to the second bag portion and the third bag portion and at a position aligned with the fourth electrode in a direction orthogonal to the circumferential direction.

11. The biological information measurement device according to claim 1, wherein
the support member is a curler curved along a circumferential direction of the measurement target site of the subject, and
the belt portion is wound around an outer circumferential side of the curler.

12. The biological information measurement device according to claim 11, wherein the curler is joined to the belt portion.

13. The biological information measurement device according to claim 1, wherein the support member constitutes a part of the belt portion.a part of the belt portion.
